(19) United States
(12) Patent Application Publication
(10) Pub. No.: US 2020/0376245 A1
(43) Pub. Date: Dec. 3, 2020

(54) Mechanical and Electrical Intracellular Access for Cells with Tough Cell Walls

(71) Applicant: The Board of Trustees of the Leland Stanford Junior University, Stanford, CA (US)

(72) Inventors: Abdulmalik M Obaid, Palo Alto, CA (US); Sergio Leal-Ortiz, Hayward, CA (US); Nicholas A. Melosh, Menlo Park, CA (US)

(21) Appl. No.: 16/891,652

(22) Filed: Jun. 3, 2020

Related U.S. Application Data
(60) Provisional application No. 62/856,226, filed on Jun. 3, 2019.

(51) Int. Cl. A61M 37/00; C12M 3/06

(52) U.S. Cl. CPC A61M 37/0015; A61M 2037/0061; A61M 2037/0053; C12M 23/16

(57) ABSTRACT

A high-throughput method, device and system technology is provided capable of unconstrained penetration into virtually any cell type. This technology is completely agnostic to the cargo type or size (DNA, RNA or protein), is ultra-robust due to use of stiff metals, and has a direct path to scalabillity. This technology will serve as an effective method of intracellular delivery. In addition, this device is reusable and capable with working with all cell types, regardless of cell stiffness, and is potentially capable of penetrating into the nucleus of a cell. An intra-cellularly delivery device with an elongated structure with an ultra-sharp tip of less than 10 nanometers enables this technology whereby intracellular access is gained with little to no observable deformation of the cell walls. This dramatically increases the likelihood of cell survival and successful delivery.

### CROSS-REFERENCE TO RELATED APPLICATIONS

This application claims priority from US Provisional Patent Application 62/856226 filed Jun. 3, 2019, which is incorporated herein by reference.

### STATEMENT OF GOVERNMENT SPONSORED SUPPORT

This invention was made with Government support under contract 1224646100QCASC awarded by the National Institutes of Health. The Government has certain rights in the invention.

### FIELD OF THE INVENTION

This invention relates to intracellular delivery. In particular, the invention relates to delivery to tough to access cells, such as plant pollen.

### BACKGROUND OF THE INVENTION

Cellular engineering is one of the most important questions in the last decade. A plethora of techniques have been used to deliver modifying agents into living cells, such as viral vectors, lipid mediated delivery and electroporation. All of these different techniques can transfer different types of cargo in and to the cells with different efficiencies and cell viability. Suboptimal cell survival is due to perturbation to genomic expression and mechanical damage. For cells with stiff cell walls, such as plant pollen, conventional methods such as electroporation or microinjection are insufficient to deliver into the cell. Therefore, Agrobacterium-mediated gene transfer has been the primary option for plant cells. However, this technique is genotype dependent, and adapting to different cell types has been slow and costly. Other alternatives include direct DNA transfer methods, such as particle gun bombardment of the target tissue. However, the high perturbation to the cell membrane affects the survival of the cell and changes mRNA expression. Additionally, most crop varieties are incompatible to delivery of CRISPR-Cas9 by standard methods such as Agrobacterium-mediated transformation or particle gun bombardment.

U.S. Pat. No. 6,686,299 teaches a nanosyringe using a syringe architecture with tip sizes on the order of several hundreds of nanometers. Such a tip size is too large to apply to cells with tough cell walls. It is noted, in general, the art has no reports of successfully interfacing with cells with tough cell walls using devices like syringe needles in U.S. Pat. No. 6,686,299 because of their relatively large size scales (>100 nm). Using devices at these sizes likely leads to cell wall rupture and cell death, both due to the initial penetration, as well as due the volume added from the device as cells with tough cell walls are especially resistant to expanding volume. In U.S. Pat. No. 6,686,299 insertion of fluid volume into the cell is accomplished utilizing a hollow syringe through which the fluid volume is pumped.

The present invention addresses these problems in the art by providing combined mechanical and electrical access and intracellular delivery into cells with tough cell walls.

### SUMMARY OF THE INVENTION

In this invention, a high-throughput method, device and system technology is provided capable of unconstrained penetration into virtually any cell type. This technology is completely agnostic to the cargo type or size (DNA, RNA or protein), is ultra-robust due to use of stiff metals, and has a direct path to scalability. This technology will serve as an effective method of intracellular delivery. In addition, this device is reusable and capable with working with all cell types, regardless of cell stiffness, and is potentially capable of penetrating into the nucleus of a cell.

An intra-cellularly delivery device is provided distinguishing a base and an elongated structure starting from the base and ending in an ultra-sharp tip which are made from a single metal structure. Examples of the single metal structure are Tungsten, Platinum, Iridium, or a combination thereof.

The base has a base width ranging from 5 micrometers to 250 micrometers. The elongated structure has a length ranging from 5 micrometers to 500 micrometers. The ultra-sharp tip has a diameter of less than 10 nanometers for over a length ranging from 10 micrometers to 50 micrometers. The elongated structure has an insulated surface except for the surface over a length of 1 micrometers to 100 micrometers measured from the end of the ultra-sharp tip where the elongated structure is not insulated, but conductive. The insulated surface has a thickness ranging from 10 nanometers to 500 nanometers. In one embodiment, the elongated structure is shaped according to a Gabriel's horn.

A method of making an intra-cellularly delivery device is provided following the steps of:
Etching a metallic wire with a length ranging from 5 micrometers to 500 micrometers partially submerged in a basic solution until the metallic wire breaks at the meniscus forming a metallic wire with an ultra-sharp tip. The ultra-sharp tip has a diameter of less than 10 nanometers.
Coating the surface of the etched metallic wire with an insulating material. The coating forms an insulated surface with a thickness ranging from 10 nanometers to 500 nanometers.
Etching back at least a portion of the coated ultra-sharp tip. This etching back results in a conductive ultra-sharp tip over a length ranging from 10 micrometers to 50 micrometers.

A high-throughput microfluidic system is provided for electro-phonetical delivery of cargo into a cell. The system includes the intra-cellularly delivery devices for the electro-phoretical delivery of the cargo into the cell. The system could have a big cargo reservoir, but in another embodiment could also be an individual cargo reservoir.

Embodiments of the invention have at least the following advantages:
Due to the protective effect of tough cell walls, such as pollen cell walls, direct cell injection or transfection methods, such as microinjection, lipofection or to electroporation, are not usually suitable for intact plant cells. The major advantage over typical techniques is that embodiments of this technology can penetrate into tough cells such as pollen and can be integrated into an array format.
Intracellular access is gained with little to no observable deformation of the cell walls. This dramatically increases the likelihood of cell survival and successful delivery.
Electrical readout and stimulation through the electrodes allow for enhanced delivery of cargo, as well as simultaneous intracellular electrical recording.
Embodiments of his technology grant simultaneous electrical and mechanical control of individual cells.
Embodiments of his technology can be completely automated and wouldn't require specialized expertise to operate it.

### BRIEF DESCRIPTION OF THE DRAWINGS

FIG. 1 shows the intra-cellularly delivery device according to an exemplary embodiment of the invention.

FIG. 2 shows according to an exemplary embodiment of the invention the method of making the intra-cellularly delivery device.

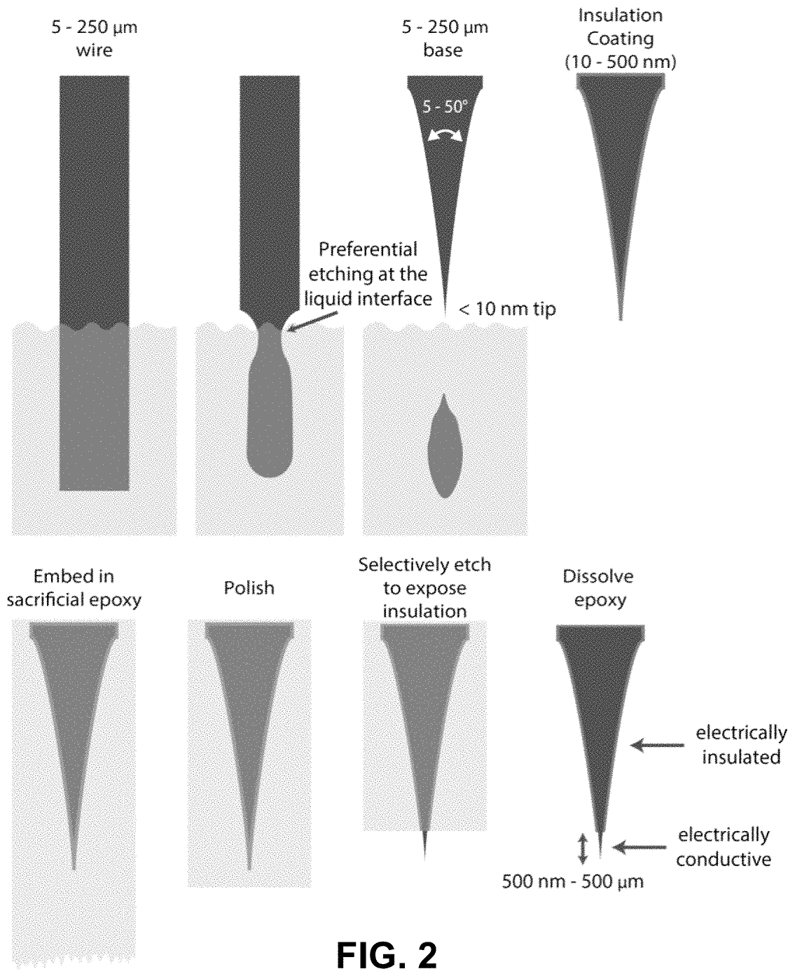

FIG. 3 shows according to an exemplary embodiment of the invention intra-cellularly delivery devices (also referred to as ultra-sharp electrodes) integrated with a microfluidic system. A line of cells is pushed (positive pressure) into a chamber with several conductive holes, slightly smaller than the cell size. A negative pressure is used to temporarily fix the cells in place, and the intra-cellularly delivery devices with their ultra-sharp tips are driven into the cells until the cells are penetrated. An electrical field is applied between the conductive tip part of the ultra-sharp electrodes and the conductive holes, and electrophoretically driving a desired cargo, between the ultra-sharp electrodes and the conductive hole, into the cell. In one embodiment, the system could have a big cargo reservoir, but in another embodiment could also be an individual cargo reservoir.

FIG. 4 shows according to an exemplary embodiment of the invention in inset A an example of intra-cellularly delivery device similar to device 100 in FIG. 1, in inset B a successful penetration of the ultra-sharp tip of the intra-cellularly delivery device into a mouse embryo with no observable deformation to the cell wall, and in insets C-D a sample penetration into a corn pollen with little to no observable deformation to the cell wall. The fluorescent center of the pollen is thought to be the nucleus.

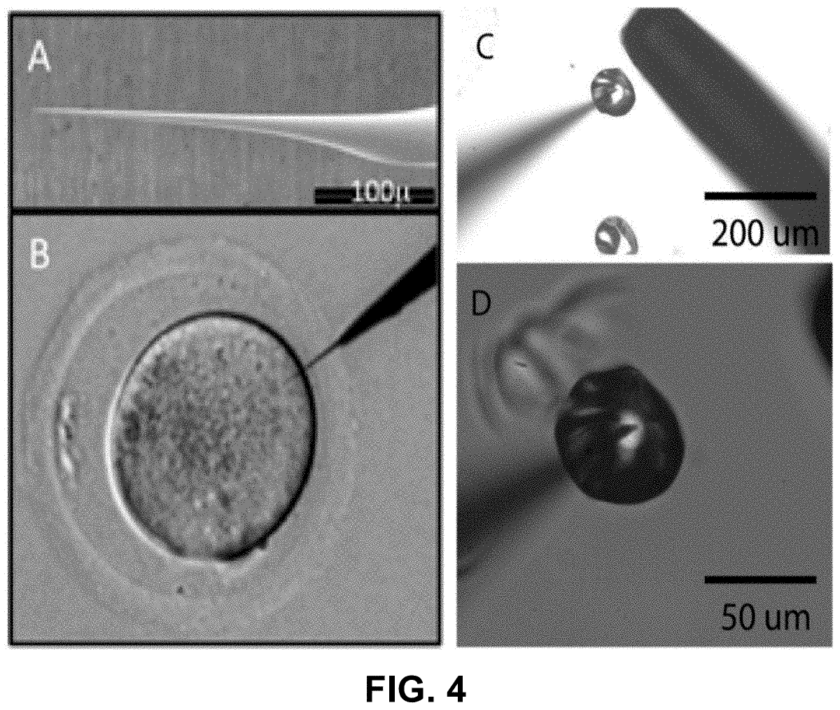

### DETAILED DESCRIPTION

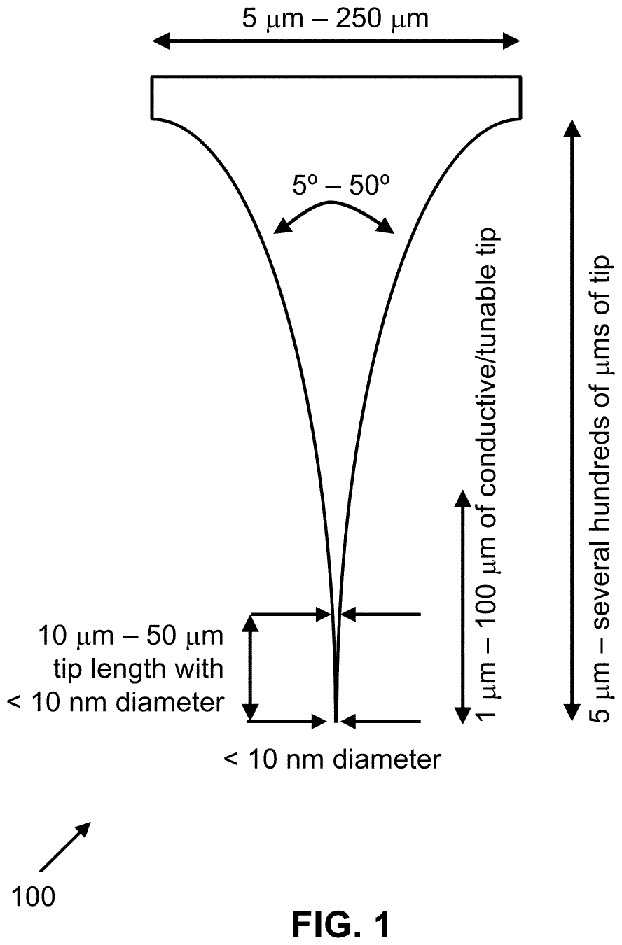

To non-destructively penetrate cells with stiff cell walls for successful delivery, an atomically sharp tip is required. FIG. 1 shows an intra-cellularly delivery device 100, which is also referred to as ultra-sharp electrode. Device 100 distinguishes a base with a base width ranging from 5 micrometers to 250 micrometers. Extending from the base is an elongated structure which ends in an ultra-sharp tip. The elongated structure has a length ranging from 5 micrometers to 500 micrometers . At the end of the elongated structure is an ultra-sharp (atomically) tip section with a diameter of less than 10 nanometers and ranging over a length of 10 micrometers to 50 micrometers. Near the tip-end of the ultra-sharp, the elongated structure is conductive over a length of 1 micrometer to 100 micrometers.

In the example shown in FIG. 1, the elongated structure is shaped elongated structure is shaped according to a Gabriel's horn. Geometry variations to this shape are part of this invention. The base and the elongated structure are one single metal structure, preferably Tungsten, Platinum, Iridium, or a combination thereof. The elongated structure can further be coated with a 1-10 nanometers thick coating to via chemical vapor deposition if needed as long as the conductive tip section remains in place or exposed.

Intra-cellularly delivery device 100 is fabricated primarily through an electro-chemical sharpening process. The ultra-sharp tip can be controllably made to have specific geometries, whether long and narrow, or sharp and broad. For some applications, there is a need for a very sharp and broad tip might be needed to successfully penetrate through the cell wall. In other applications, a sharp, long and narrow tip is needed to penetrate through the cell membrane and reach a specific target inside the cell, such as the nucleus or sperm. These different application needs fall within the dimensions as shown in FIG. 1.

The steps for electrochemical sharpening of intra-cellularly delivery device 100 are generally as follows:

1. A metallic wire (e.g. Tungsten or Platinum-Iridium) is submerged in a basic solution (e.g. potassium hydroxide, alkali chloride solution can be used for Tungsten and Platinum-Iridium, respectively), and a potential difference (up to 10 Volts direct current, or up to 20 Volts alternating) is applied between this metallic wire and a cathode.

2. The submerged portions of the metallic wire begin to etch away, but the wire etches more rapidly at the meniscus of the solution due to the solution/air interface. The metallic wire etches at the meniscus until the weight of the submerged portion of the metallic wire is greater than the tensile force at the etching point.

3. The metallic wire breaks at the meniscus and an ultra-sharp tip is then created. As soon as the metallic wire breaks, the applied potential must be stopped immediately (<100 ms resolution) because etching after this point will dull the tip further. To do this, one can use a circuit to measure the resistance between the metallic wire and the cathode, and detect when a significant change in resistance occurs, which is coincident with the breaking point. This is critical in maintaining <100 nm ultra-sharp tips, which is an enabling feature for use in intracellular access of tough-cell walled cells. An additional step of a brief ion-milling step could be considered to increase the sharpness of the tips in case the electrochemical circuit cannot cutoff the applied potential fast enough.

4. For post etching of the metallic wires, the metallic wires can be coated with an insulating material (e.g. Al₂O₃, SiO₂, HfO₂, or the like), and controllably exposed at the tip. A thin, conformal electrical insulation coating (i.e. alumina) is deposited through a chemical vapor or atomic-layer deposition process (CVD, ALD respectively).

5. Then, the coated metallic wire can be embedded in a sacrificial material, such as with an optically transparent epoxy.

6. The coated metallic wire embedded with epoxy can then be polished using a conventional grinding and polishing tool to create a flat surface. Care is to be taken to ensure that only the sacrificial epoxy is polished, and not the sharp tip. This can be confirmed with optical inspection.

7. The sacrificial epoxy can then be controllably etched back to expose a specific amount of the tip, with <100 nm resolution. This can be done by a variety of etching methods, such as etching the epoxy with a dry-etching process to selectively etch the epoxy without attacking the metallic sharp tip. This etch will control the overall length of the sharp tip that is electrically exposed (conductive). Once the desired tip-exposure is reached through the dry-etch process, an additional selective etch exposes the thin insulation coating from step 4. This can be done through a wet process to remove the thin insulation coating (i.e. ceramic etchant to remove alumina) without damaging the ES tip and without exposing the rest of the insulation around the wire.

8. Finally, the sacrificial epoxy can be etched completely, leaving an insulated wire with a conductive ultra-sharp tip.

While the method is described for processing a single metallic wire into an intra-cellularly delivery device 100, this technology can be scaled to arrays of hundreds to thousands of ultra-sharp electrodes or intra-cellularly delivery devices 100. Electro-sharpened wire fabrication is extremely reproducible and can be done in a batch process.

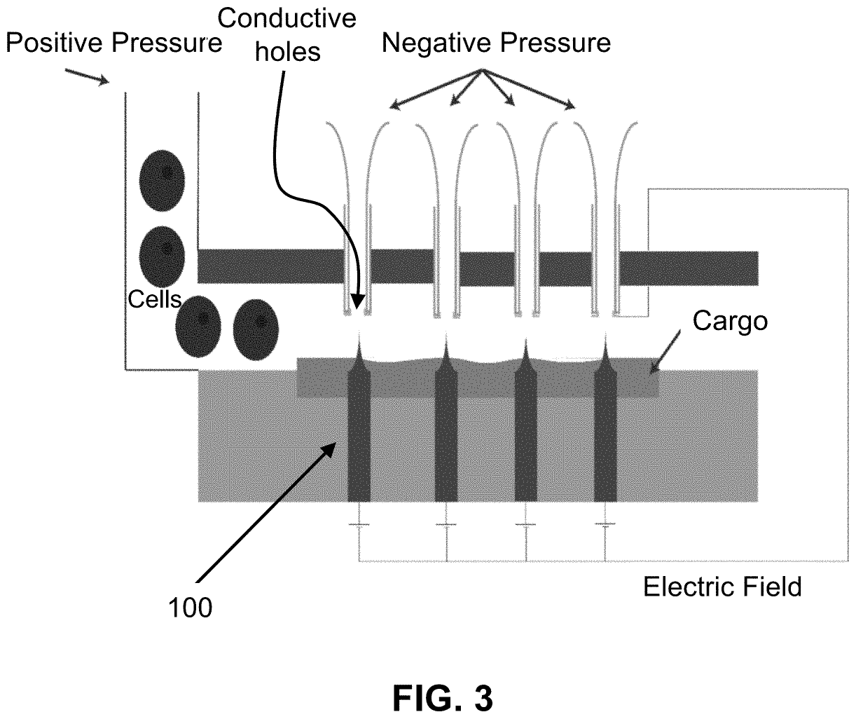

In a high-throughput system, intra-cellularly delivery devices 100 can be integrated with microfluidics as shown in FIG. 3 to sequentially penetrate and deliver into a stream of cells. The cells are first pushed via a positive pressure (100-1000 hPa) into a chamber with several holes, slightly smaller than the cell size. A negative pressure (100-1000 hPa) is used to temporarily fix them in place, and the ultra-sharp tips are physically driven into the cells until they are penetrated. With a small incision into the cell wall, a small electrical field (e.g. a charge that is negatively biased, voltages 1-100 mV are used to drive the cargo in) is applied between the probe and the hole containing the cell to electrophoretically drive the desired cargo into the cell.

The use of a conductive tip of the intra-cellularly delivery device 100 allows for both applying and recording electrical current. Electrical recording can be used as a means to confirm intracellular access. Typically, a large change in electrical impedance is observed when an electrode is inside the cell.

The access ports are or can also be electrically conductive, opening opportunities for temporally overlapping fields to target a specific location inside the cell. This could allow for enhanced cargo delivery to a specific location inside the cell.

Embodiments of the invention can be applied or used in various ways such as genetic modification of any cell type, especially highly specialized cells with low cell counts and very tough cells (i.e. pollen) previously impossible to modify with conventional techniques. The inventors have been successful in penetrating maize and corn pollen. Upon confirming successful delivery, this is of significant commercial interest because it allows for the direct transfection of pollen with high efficiency, eliminating the need for costly work with plant tissue culture. The procedure of tissue regeneration from protoplasts and the identification of gene-edited plants from bombarded embryos can be very costly and laborious. So far, these transgene-independent techniques are only feasible with very few plant species and varieties. Being able to directly modify pollen significantly reduces the cost of genetic modification of plants, and allows the use rapid use of CRISPR-Cas9 and other genetic modification techniques into virtually any plant type. Current techniques are primarily genotype dependent. The inventors have also been to successful in penetrating into mouse embryo, with no observable deformation to the cell wall. Microinjection typically causes significant deformation of the cell wall. Application of electric fields to deliver cargo, as well as control the rate of delivery.

What is claimed is:

1. An intra-cellularly delivery device, comprising:
(a) a base with a base width ranging from 5 micrometers to 250 micrometers; and
(b) an elongated structure starting from the base and ending in an ultra-sharp tip, wherein the elongated structure has a length ranging from 5 micrometers to 500 micrometers , wherein the ultra-sharp tip has a diameter of less than 10 nanometers for over a length ranging from 10 micrometers to 50 micrometers, wherein the elongated structure has an insulated surface except for the surface over a length of 1 micrometers to 100 micrometers measured from the end of the ultra-sharp tip where the elongated structure is not insulated, but conductive, and wherein the base and the elongated structure are one single metal structure.

2. The intra-cellularly delivery device as set forth in claim 1, wherein the insulated surface has a thickness ranging from 10 nanometers to 500 nanometers.

2. The intra-cellularly delivery device as set forth in claim 1, wherein elongated structure is shaped according to a Gabriel's horn.

3. The intra-cellularly delivery device as set forth in claim 1, wherein the single metal structure is Tungsten, Platinum, Iridium, or a combination thereof.

4. A method of making an intra-cellularly delivery device, comprising:
(a) etching a metallic wire with a length ranging from 5 micrometers to 500 micrometers partially submerged in a basic solution until the metallic wire breaks at the meniscus forming a metallic wire with an ultra-sharp tip, wherein the ultra-sharp tip has a diameter of less than 10 nanometers;
(b) coating the surface of the etched metallic wire with an insulating material, wherein the coating forms an insulated surface with a thickness ranging from 10 nanometers to 500 nanometers; and
(c) etching back at least a portion of the coated ultra-sharp tip, wherein etching back results in a conductive ultra-sharp tip over a length ranging from 10 micrometers to 50 micrometers.

5. A high-throughput microfluidic system for electro-phoretical delivery of cargo into a cell, wherein the system comprises the intra-cellularly delivery devices as set forth in claim 1 for the electro-phoretical delivery of the cargo into the cell.
